(19) United States
(12) Patent Application Publication
(10) Pub. No.: US 2024/0074765 A1
(43) Pub. Date: Mar. 7, 2024

(54) EMBOLIZATION DEVICES INCLUDING BRAIDED WIRES, METHODS OF MAKING AND USING THE SAME

(71) Applicant: Merit Medical Systems, Inc., South Jordan, UT (US)

(72) Inventors: John Oslund, South Jordan, UT (US); Kevin Dunne, South Jordan, UT (US)

(21) Appl. No.: 18/459,721

(22) Filed: Sep. 1, 2023

Related U.S. Application Data
(60) Provisional application No. 63/374,503, filed on Sep. 2, 2022.

(51) Int. Cl. A61B 17/12

(52) U.S. Cl. CPC A61B 17/12172; A61B 17/12177; A61B 2017/00867

(57) ABSTRACT

Embolization devices, including devices formed of wires or filaments, including braided wires or filaments are disclosed. An example embolization device includes a plurality of wires that are braided together to form an elongated sleeve. The elongated sleeve may define an interior region. Related systems, methods of use, and methods of manufacture are also contemplated.

### RELATED APPLICATIONS

This application claims priority to U.S. Provisional Application No. 63/374,503 filed on Sep. 2, 2022 and titled “Embolization Devices Including Braided Wires, Methods of Making and Using the Same,” which is hereby incorporated by reference in its entirety.

### TECHNICAL FIELD

The present disclosure relates to embolization devices, including embolization devices comprised of one or more wires that may be braided or woven.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings. Identical reference numerals refer to identical or similar elements or features in different views or embodiments shown in the drawings.

FIG. 1 is a pictorial view of an embolization device, according to an embodiment.

FIG. 2 is a pictorial view of an embolization device that includes an at least partially occupied interior region, according to an embodiment.

FIG. 3 is a pictorial view of an embolization device that includes fibers, according to an embodiment.

FIG. 4 is a pictorial view of an embolization device that includes at least one end cap, according to an embodiment.

FIG. 5 is a pictorial view of an embolization device reversibly attached to a medical device, according to an embodiment.

FIG. 6A is a side view of an embolization device in a deployed configuration, according to an embodiment.

FIG. 6B is a top plan view of an embolization device in a deployed configuration, according to an embodiment

FIG. 6C is a top plan view of an embolization device, according to an embodiment.

FIG. 7 is a pictorial view of a first example method of shaping the elongated sleeves of embolization devices such that the elongated sleeves exhibit a selected shape when in the deployed configuration, according to different embodiments.

FIG. 8 is a pictorial view of a second example method of shaping the elongated sleeves of embolization devices such that the elongated sleeves exhibit a selected shape when in the deployed configuration, according to different embodiments.

FIG. 9A is a partial cross-sectional view of a pre-deployment step of a method of using any of the embolization devices disclosed herein, according to an embodiment.

FIG. 9B is a partial cross-sectional view of a deployment step of a method of using any of the embolization devices disclosed herein, according to an embodiment.

### DETAILED DESCRIPTION

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The present disclosure includes embodiments of embolization devices including braided wires, methods of making the same, and methods of using the same. An example embolization device includes a plurality of wires that are braided together to form an elongated sleeve. The elongated sleeve may define an interior region. The embolization devices disclosed herein are improvements over conventional embolization coils. For example, at least some conventional embolization coils include a single wire that is helically wrapped. Such conventional embolization coils exhibit relatively limited surface area. The rate at which blood clots on the embolization wires is dependent, at least in part, on the surface area of the conventional embolization coils and how densely they are packed into a blood vessel. As such, the relatively limited surface area of conventional embolization coils limits the rate at which blood clots on the conventional embolization coils. Further, such conventional embolization coils exhibit relatively limited expansion when dispensed from a catheter into a body. The ability of the conventional embolization coils to occupy a volume of the body depends, at least in part, on the ability of the conventional embolization coils to expand. Any treatment (e.g., for an aneurysm) that uses the conventional embolization coils may be ineffective if the embolization coils do not occupy a sufficient volume of the site being embolized. To remedy these deficiencies of conventional embolization devices, users of such conventional embolization devices often dispose a plurality of conventional embolization coils into the body to increase the surface area on which the blood may clot and/or to ensure that a volume is completely occupied by the conventional embolization coils.

The embolization devices disclosed herein include a plurality of wires that are braided together. Braiding the wires together significantly increases the surface area of the embolization devices compared to at least some conventional embolization coils when the embolization devices and the conventional embolization coils exhibits the same length and maximum lateral dimension. For example, braiding the wires to form the embolization devices disclosed herein forms pores between the braided wires which increases the surface area and device volume for maximum flow disruption of the embolization devices disclosed herein. The conventional embolization coils do not include such pores nor the ability for the base structure to expand radially and, thus, do not exhibit the increased surface area. Further, braiding the wires together allows the embolization devices disclosed herein to expand more than conventional embolization coils when dispensed from a catheter. For example, braiding the wires together allows the maximum lateral dimension to increase more when dispensed from the catheter than conventional embolization coils. In other words, the embolization devices disclosed herein are an improvement over conventional embolization devices at least because conventional embolization devices have a fixed diameter both during delivery and after deployment while the embolization devices disclosed herein can collapse to a small diameter during delivery and hen expand to a larger diameter after deployment to increase the surface area, volume, and cross-sectional coverage. It is currently believed that the increased surface area and expansion of the embolization devices disclosed herein relative to conventional embolization coils may allow a single embolization device to perform the task of a multitude of similarly sized conventional embolization coils.

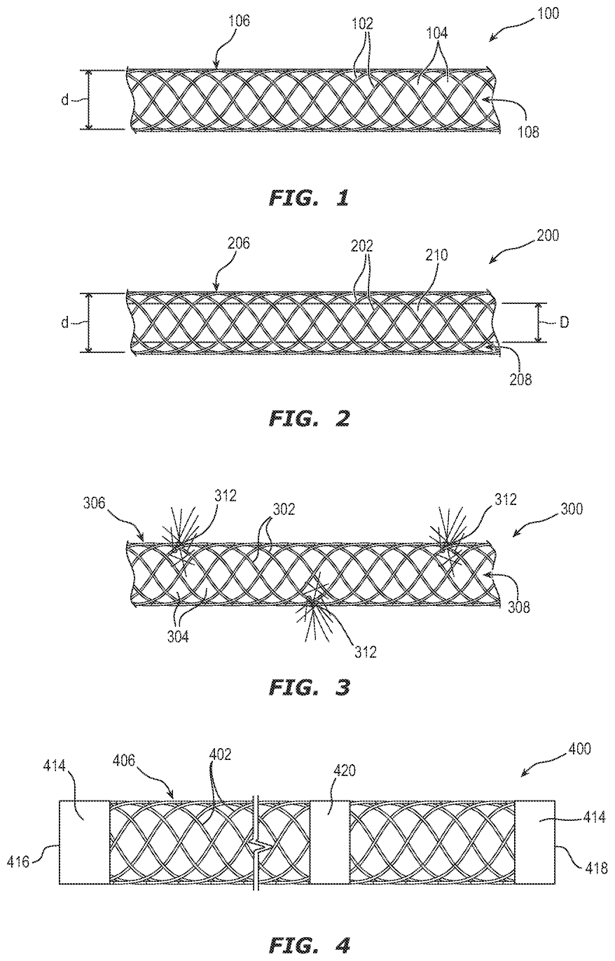

FIG. 1 is a pictorial view of an embolization device 100, according to an embodiment. The embolization device 100 includes a plurality of wires 102 braided together. In other words, the embolization device 100 includes a plurality of overlapping and intertwined wires 102. The wires 102 define a plurality of pores 104 therebetween. The wires 102 and the pores 104 collectively form an elongated sleeve 106 defining an interior region 108. In the illustrated embodiment, the embolization device 100 consists of the elongated sleeve 106 However, as will be discussed in more detail below, the embolization device 100 may include components in addition to the elongated sleeve 106. The wires 102 and the pores 104 of the embolization device 100 allows the embolization device 100 to exhibit a relatively large surface area and expanded volume to increase cross-sectional coverage in the blood vessel which, in turn, allows the embolization device to promote blood clotting. The wires 102 and the pores 104 of the embolization device 100 also allows the elongated sleeve 106 to exhibit a relatively large deployed configuration, as will be discussed in more detail below.

The plurality of wires 102 of the elongated sleeve 106 may include 4 or more wires, such as about 5 or more wires, about 10 or more wires, about 15 or more wires, about 20 or more wires, about 30 or more wires, about 40 or more wires, about 50 or more wires, about 60 or more wires, about 70 or more wires, about 80 or more wires, about 90 or more wires, about 100 or more wires, about 110 or more wires, about 120 or more wires, about 130 or more wires, about 140 or more wires, about 150 or more wires, about 175 or more wires, about 200 or more wires, about 250 or more wires, about 300 or more wires, about 350 or more wires, about 400 or more wires, about 450 or more wires, about 500 or more wires, or in ranges of about 5 wires to about 15 wires, about 10 wires to about 20 wires, about 15 wires to about 30 wires, about 20 wires to about 40 wires, about 30 wires to about 50 wires, about 40 wires to about 60 wires, about 50 wires to about 70 wires, about 60 to about 80 wires, about 70 to about 90 wires, about 80 to about 100 wires, about 90 to about 110 wires, about 100 wires to about 120 wires, about 110 wires to about 130 wires, about 120 wires to about 140 wires, about 130 wires to about 150 wires, about 140 wires to about 175 wires, about 150 wires to about 200 wires, about 175 wires to about 250 wires, about 200 wires to about 300 wires, about 250 wires to about 350 wires, about 300 wires to about 400 wires, about 350 wires to about 450 wires, or about 400 wires to about 500 wires. Generally, increasing the number of wires 102 that form the elongated sleeve 106 increases the strength (e.g., inhibits collapse) of and increases the surface area of the elongated sleeve 106. However, increasing the number of wires 102 that form the elongated sleeve 106 also increases the amount of overlap between the wires 102. Increasing the amount of overlap between wires 102 may also increase the net friction acting on the elongated sleeve 106 due to friction between the wires 102. The increased friction may limit the maximum lateral dimension d of the elongated sleeve 106 (e.g., outer diameter when the elongated sleeve 106 is cylindrical) by limiting, for example, how small of an maximum lateral dimension the elongated sleeve 106 may exhibit after braiding, limiting how much the maximum lateral dimension d may be drawn down (as discussed in more detail below), and how much the maximum lateral dimension d may increase when the elongated sleeve 106 switches from the elongated configuration to the deployed configuration. As such, the number of wires 102 that form the elongated sleeve 106 may be selected based on balancing these factors and/or based on the application of the embolization device 100. For instance, the number of wires 102 may be relatively small when the embolization device 100 is required to have a relatively small maximum lateral dimension d (e.g., the embolization device 100 is configured to be disposed in a small artery or vein or used with a small catheter). Alternatively, the number of wires 102 may be relatively large when the maximum lateral dimension d of the elongated sleeve 106 is not limited, when a high rate of blood clotting is required, or relatively large compressive forces are expected to be applied to the elongated sleeve 106. Also, the number of wires 102 may be limited based on the manufacturing device that forms the embolization device 100. For example, certain braiding devices may be configured to hold a maximum number of distinct wires thereby limiting the number of wires 102 that may form the embolization device 100. The number of wires 102 may also be selected based on the average maximum lateral dimension of the wires 102. For example, a fewer number of wires 102 may be used as the maximum lateral dimension is increased and vice versa. The number of wires 102 may also be selected based on the desired density and structure of the elongated sleeve 106. For example, the elongated sleeve 106 may include fewer wires 102 when a sparcer structure (i.e., less dense) structure is desired and vice versa. The number of wires 102 may also be selected to optimize the properties (e.g., stiffness) of the elongated sleeve 106.

The wires 102 may exhibit a maximum lateral dimension (e.g., outer diameter) that is about 10 μm or greater, about 15 μm or greater, about 20 μm or greater, about 25 μm or greater, about 30 μm or greater, about 40 μm or greater, about 50 μm or greater, about 60 μm or greater, about 70 μm or greater, about 80 μm or greater, about 90 μm or greater, about 100 μm or greater, about 125 μm or greater, about 150 μm or greater, or in ranges of about 10 μm to about 20 μm, about 15 μm to about 25 μm, about 20 μm to about 30 μm, about 25 μm to about 40 μm, about 30 μm to about 50 μm, about 40 μm to about 60 μm, about 50 μm to about 70 μm, about 60 μm to about 80 μm, about 70 μm to about 90 μm, about 80 μm to about 100 μm, about 90 μm to about 125 μm, or about 100 μm to about 150 μm. Generally, increasing the maximum lateral dimension of the wires 102 increasing the strength of the wires 102 and increase the overall surface area of the elongated sleeve 106. However, increasing the maximum lateral dimension of the wires 102 also increases the overlap between the wires 102. Thus, the maximum lateral dimension of the wires 102 may be selected based on balancing these factors. Also, the maximum lateral dimension of the wires 102 may be selected based on the desired maximum lateral dimension d of the elongated sleeve 106 and/or the desired number of wires 102 in the elongated sleeve 106. For example, decreasing the maximum lateral dimension of the wires 102 may allow for a decreased maximum lateral dimension d of the elongated sleeve 106 or an increase in the number of wires 102 in the elongated sleeve 106 since the smaller maximum lateral dimension of the wires 102 decreases overlap between the wires 102.

In an embodiment, as shown, the embolization device 100 includes a single layer of braided wires 102. In an embodiment, the embolization device 100 includes a plurality of layers of braided wires 102, such as 2, 3, 4, 5, 6, 7, 8, 9, or 10 or more layers. In such an embodiment, the plurality of layers of the wires 102 may be concentrically arranged. The plurality of layers of the wires 102 may increase the surface area of the embolization device 100 compared to a similar embolization device 100 that only includes a single layer of the wires 102. However, the plurality of layers of the wires 102 may increase the complexity and difficulty of forming the embolization device 100 compared to a similar embolization device 100 that only includes a single layer of wires 102.

In an embodiment, each of the wires 102 exhibits the same or substantially the same maximum lateral dimension (e.g., the maximum lateral dimensions differ by at most ±10%). In an embodiment, at least some of the wires 102 exhibit significantly different maximum lateral dimensions (e.g., the maximum lateral dimensions differ by more than ±10%). In such an embodiment, at least some of the wires 102 may exhibit a maximum lateral dimensions that vary by about 10% or more, about 20% or more, about 40% or more, about 60% or more, about 80% or more, about 100% or more, about 125% or more, about 150% or more, about 175% or more, about 200% or more, about 250% or more, about 300% or more, or in ranges of about 10% to about 40%, about 20% to about 60%, about 40% to about 80%, about 60% to about 100%, about 80% to about 125%, about 100% to about 150%, about 125% to about 175%, about 150% to about 200%, about 175% to about 250%, or about 200% to about 300%. In such an embodiment, the significantly different maximum lateral dimensions may allow for more control over the properties (e.g., stiffness, density, strength, lateral expansion, etc.) of the elongated sleeve 106 and provide greater manufacturing options for the elongated sleeve 104. For example, the elongated sleeve 106 may be formed with at least one first wire exhibiting a first maximum lateral dimension and at least one second wire exhibiting a second maximum lateral dimension that is smaller than the first maximum lateral dimension. The first maximum lateral dimension of the at least one first wire may increase the strength of the elongated sleeve while the second maximum lateral dimension minimized overlap between the first and second wires.

As previously discussed, the elongated sleeve 106 may exhibit a maximum lateral dimension d. The maximum lateral dimension d may be an outer diameter of the elongated sleeve 106 when the elongated sleeve 106 exhibits a generally cylindrical shape. The maximum lateral dimension d of the elongated sleeve 106 may be selected to be about 0.5 mm or less, about 1 mm or less, about 2 mm or less, about 3 mm or less, about 4 mm or less, about 0.05 mm or greater, about 0.75 mm or greater, about 0.1 mm or greater, about 0.15 mm or greater, about 0.2 mm or greater, about 0.3 mm or greater, about 0.4 mm or greater, about 0.5 mm or greater, about 1 mm or greater, about 2 mm or greater, about 3 mm or greater, or in ranges of about 0.05 mm to about 0.1 mm, about 0.075 mm to about 0.15 mm, about 0.1 mm to about 0.2 mm, about 0.15 mm to about 3 mm, about 0.2 mm to about 0.4 mm, about 0.3 mm to about 0.5 mm, about 0.4 mm to about 0.75 mm, about 0.5 mm or about 1 mm, about 0.75 mm to about 1.25 mm, about 1 mm to about 1.5 mm, about 1.25 mm to about 1.75 mm, about 1.5 mm to about 2 mm, about 1.75 mm to about 2.5 mm, about 2 mm to about 3 mm, about 2.5 mm to about 3.5 mm, about 3 mm to about 4 mm, about 3.5 mm to about 5 mm, about 4 mm to about 6 mm, or about 5 mm to about 7.5 mm. As previously discussed, the maximum lateral dimension d of the elongated sleeve 106 may be selected based on the number of wires 102 that form the elongated sleeve 106 and the maximum lateral dimension of the wires 102. Also, the maximum lateral dimension d of the elongated sleeve 106 may be selected based on the size of the lumen or cavity of the body that the embolization device 100 is configured to be disposed in and the size of the catheter that is configured to deliver the embolization device 100 into the body.

In some embodiments, it may be difficult to form the elongated sleeve 106 that exhibits a relatively small maximum lateral dimensions d (e.g., maximum lateral dimensions that are smaller than 3 mm) using a braiding machine, such as when the elongated sleeve 106 is formed with 70 or more wires 102. In such an embodiment, after forming the elongated sleeve 106 with the braiding machine, the maximum lateral dimension d of the elongated sleeve 106 may be reduced by stretching the elongated sleeve 106. The elongated sleeve 106 may be stretched since the elongated sleeve 106 is formed from braided wires 102. Stretching the elongated sleeve 106 draws down the elongated sleeve 106 thereby decreasing the maximum lateral dimension d of the elongated sleeve 106. The elongated sleeve 106 may be stretched by gripping two portions of the elongated sleeve 106 that are spaced from each other (e.g., portions of the elongated sleeve 106 at or near the terminal ends thereof) and moving the two portions of the elongated sleeve 106 away from each other. Stretching the elongated sleeve 106 may be used to reduce the maximum lateral dimension d of the elongated sleeve 106 by about 1% or more, about 5% or more, about 10% or more, about 15% or more, about 20% or more, about 25% or more, about 30% or more, about 40% or more, about 50% or more, about 60% or more, about 70% or more, about 80% or more, about 90% or more, about 100% or more, about 125% or more, about 150% or more, about 175% or more, about 200% or more, about 250% or more, about 300% or more, or in ranges of about 1% to about 10%, about 5% to about 15%, about 10% to about 20%, about 15% to about 25%, about 20% to about 30%, about 25% to about 40%, about 30% to about 50%, about 40% to about 60%, about 50% to about 70%, about 60% to about 80%, about 70% to about 90%, about 80% to about 125%, about 100% to about 150%, about 125% to about 175%, about 150% to about 200%, about 175% to about 250%, or about 200% to about 300%. It is noted that stretching the elongated sleeve 106 may increase the length of the elongated sleeve 106, increase the size of the pores 104, and decrease the volume of the interior region 108.

The elongated sleeve 106 may exhibit any suitable shape. In an embodiment, as illustrated, the elongated sleeve 106 exhibits a generally cylindrical shape. The generally cylindrical shape of the elongated sleeve 106 may simplify manufacturing of the elongated sleeve 106. However, forming the elongated sleeve 106 from braided wires 102 allows the elongated sleeve 106 to exhibit a variety of shapes. Examples of shapes that the elongated sleeve 106 may exhibit include a generally conical shape or other shapes where the maximum lateral dimension d of the elongated sleeve changes along at least a portion of the length thereof. In an example, the non-cylindrical shape of the elongated sleeve 106 may be formed using the braiding machines, for instance, by varying the tension of the wires 102 in the braiding machine or by varying the speed at which the braided portions of the sleeve 106 move away from the source of the wires 102. In an example, the non-cylindrical shape of the elongated sleeve 106 may be formed by stretching only selected portions of the elongated sleeve.

The wires 102 may be formed from any suitable bio-compatible material. In an example, the wires 102 may be formed from stainless steel, titanium-cobalt-chromium alloys, other biocompatible metals, alumina, bioglass, hydroxyapatite, other biocompatible ceramics, medical-grade silicone, polyvinylchloride, polyethylene, polypropylene, other biocompatible polymers, biocompatible composites, or combinations thereof. In an example, the wires 102 may be formed from one or more shape memory materials, such as nitinol, gold-cadmium alloys, cobalt-nickel-aluminum alloys, cobalt-nickel-gallium alloys, copper-aluminum-nickel alloys, copper-zinc alloys, copper-tin alloys, iron-magnesium-silicon alloys, or any other suitable biocompatible shape memory material. In some embodiments, the wires 102 are formed from nitinol.

Forming the wires 102 from shape memory materials has several benefits over forming the wires 102 from non-shape memory materials. For example, many shape memory materials exhibit super-elasticity. Relying only on elastic deformation of the wires 102, the super-elasticity of the shape memory materials allows the elongated sleeve 106 to exhibit a deployed configuration larger degree of difference than an elongated configuration than if the wires 102 were formed from a non-shape memory material. Further, certain shape memory materials may exhibit a heat set shape. The heat set shape of the shape memory material allows the elongated sleeve 106 to switch between the elongated and deployed configurations even when such switching results in temporary deformation of the wires 102. It is noted that wires 102 formed from a non-shape memory material may be unable to switch between elongated and deployed configurations when such switching would result in plastic deformation of the wires 102. In an embodiment, the wires 102 are formed from one or more shape memory materials and one or more non-shape memory materials.

As previously discussed, the elongated sleeve 106 may be configured to switch between an elongated configuration and a deployed configuration. For example, the elongated sleeve 106 may exhibit an elongated configuration when the embolization device 100 is at least partially disposed in a lumen of a medical device (e.g., catheter). Dispensing the embolization device 100 from the lumen of the medical device allows the elongated sleeve 106 to switch to the deployed configuration. Switching the elongated sleeve 106 from the elongated configuration to the deployed configuration allows the elongated sleeve 106 to expand to a larger diameter after deployment. In other words, the elongated sleeve 106 is able collapse to a smaller diameter during delivery and then expand to a larger diameter after deployment. The elongated sleeve 106 in the deployed configuration may exhibit a less elongated shape (e.g., exhibit a smaller length), occupy more volume, exhibit increased cross-sectional coverage, increased surface area, and/or exhibit a larger maximum lateral dimension d compared to the elongated sleeve 106 that is in the elongated configuration. In an embodiment, the elongated sleeve 106 may exhibit the elongated configuration due to compressive forces applied to the elongated sleeve 106. The compressive forces may be applied to the elongated sleeve 106 when the elongated sleeve 106 is disposed in the lumen of the medical device. Dispensing the embolization device 100 from the lumen of the medical device removes the compressive stress applied to the elongated sleeve 106 thereby allowing the elongated sleeve 106 to switch to the deployed configuration due to the elasticity of the wires 102. Dispensing the embolization device 100 from the lumen (e.g., switching from the elongated configuration to the deployed configuration) may cause the elongated sleeve 106 to expand laterally. The lateral expansion of the elongated sleeve 104 may allow the elongated sleeve 106 to fill and/or cover and/or appose a larger area of a blood vessel than a conventional embolization device exhibiting a size and shape similar to the embolization devife 100 in the elongated configuration. When the wires 102 are formed from certain shape memory materials, heat from the body may also cause the elongated sleeve 106 to switch to a heat set shape thereof. In other words, when the wires 102 are formed from a shape memory material, the elongated sleeve 106 may not rely solely on the elasticity thereof to switch from the elongated configuration to the deployed configuration.

The interior region 108 of the embolization device 100 is substantially empty. The interior region 108 may be substantially empty, for example, when the elongated sleeve 106 is formed without using a mandrel or the mandrel used to form the elongated sleeve 106 is removed. The embolization device 108 with a substantially empty interior region 108 may exhibit increased flexibility than if the interior region 108 was at least partially occupied. The increased flexibility may facilitate changing the elongated sleeve 106 from the elongated configuration to the deployed configuration. However, the substantially empty interior region 108 may exhibit a lower strength (e.g., radial strength) and, thus, be more likely to collapse during use than if the interior region 108 was at least partially occupied. Thus, in some embodiments, the embolization devices disclosed herein may include an at least partially occupied interior region. For example, FIG. 2 is a pictorial view of an embolization device 200 that includes an at least partially occupied interior region 208, according to an embodiment. FIG. 2 depicts an embodiment of an embolization device that resembles the embolization device 100 described above in certain respects. Accordingly, like features are designated with like reference numerals, with the leading digits incremented to “2.” For example, the embodiment depicted in FIG. 2 includes a plurality of wires 202, an elongated sleeve 206, and an interior region 208 that may, in some respects, resemble the plurality of wires 102, the elongated sleeve 106, and the interior region 108, respectively, of FIG. 1. Relevant disclosure set forth above regarding similarly identified features thus may not be repeated hereafter. Moreover, specific features of the embolization device 200 and related components shown in FIG. 1 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the embolization device 200 and related components depicted in FIG. 2. Any suitable combination of the features, and variations of the same, described with respect to the embolization device 100 and related components illustrated in FIG. 1 can be employed with the embolization device 200 and related components of FIG. 2, and vice versa. This pattern of disclosure applies equally to further embodiments depicted in subsequent figures and described hereafter, wherein the leading digits may be further incremented.

The embolization device 200 includes a mandrel 210 at least partially disposed in the interior region 208 of the elongated sleeve 206. The mandrel 210 provides strength to the embolization device 200 thereby preventing or at least inhibiting collapse of the elongated sleeve 206. The mandrel 210 also increases the surface area of the embolization device 200 which may thereby increasing the rate at which the embolization device 200 causes blood to clot.

In an embodiment, as shown, the mandrel 210 only occupies a portion of the interior region 208. In such an embodiment, the mandrel 210 exhibits a maximum lateral dimension D that is smaller than the maximum lateral dimension D of the mandrel 210 is substantially equal to the maximum internal diameter of the elongated sleeve 206 (e.g., the maximum lateral dimension D of the elongated sleeve 206 minus the maximum lateral dimension of the wires 202) and/or exhibits a cross-sectional shape that is different than the cross-sectional shape of the elongated sleeve 206. Only partially occupying the interior region 208 with the mandrel 210 allows a portion of the elongated sleeve 206 to partially collapse when a sufficiently large compressive load is applied thereof. Partially collapsing the elongated sleeve 206 allows the elongated sleeve 206 to conform to a surface. It is noted that the presence of the mandrel 210 prevents total collapse of the elongated sleeve 206. In an embodiment, the mandrel 210 occupies substantially all of the interior region 208. In such an embodiment, the mandrel 210 exhibits a maximum lateral dimension D that is equal to or substantially equal to the maximum lateral dimension D of the elongate sleeve 206 and exhibits a cross-sectional shape that is substantially the same as the cross-sectional shape of the elongated sleeve 206. Occupying substantially all of the interior region 208 with the mandrel 210 may thus increase the radial strength of the embolization device 200 such that the elongated sleeve 206 is substantially unable to collapse regardless of the compressive loads applied thereto.

The mandrel 210 may be disposed in the interior region 208 simultaneously with or after braiding the wires 202. Disposing the mandrel 210 in the interior region 208 simultaneously with braiding the wires 202 may include braiding the wires 202 over the mandrel 210. Braiding the wires 202 over the mandrel 210 may result in a tighter weave pattern than if the wires 202 are braided without using the mandrel 210. Disposing the mandrel 210 in the interior region 208 after braiding the wires 202 may include disposing a mandrel 210 in the interior region 208 that only partially occupies the interior region 208 to minimize friction between the wires 202 and the mandrel 210 which may make inserting the mandrel 210 into the interior region 208 difficult. However, the elongated sleeve 206 may be stretched after disposing the mandrel 210 in the interior region 208 such that the mandrel 210 substantially completely occupies all of the interior region 208.

The mandrel 210 may be formed from any of the biocompatible materials disclosed herein. In an example, the mandrel 210 may be formed from substantially the same material as the wires 202 to prevent issues associated with different coefficients of thermal expansion. In an example, the mandrel 210 may be formed from a shape memory material (e.g., Nitinol, shape memory polymer, etc.) such that the mandrel 210 may exhibit a heat set shape. The heat set shape of the mandrel 210 may facilitate switching the elongated sleeve 206 from the elongated configuration to the deployed configuration even if such switching from the deployed configuration to the elongated configuration involves plastic deformation. In an embodiment, the mandrel 210 may be formed from a material that is equally elastic or more elastic (e.g., exhibits a Young's modulus that is smaller) than the material that forms the wires 202 to minimize the effect the mandrel 210 has on switching the elongated sleeve 206 from the elongated configuration to the deployed configuration. In an embodiment, the mandrel 210 may be formed from stainless steel, titanium, or another biocompatible material.

The embolization devices disclosed herein may include the elongate sleeve and other components instead of or in addition to the mandrel. For example, FIG. 3 is a pictorial view of an embolization device 300 that includes fibers 312, according to an embodiment. Except as otherwise disclosed herein, the embolization device 300 is the same as or substantially similar to any of the embolization devices disclosed herein. For example, the embolization device 300 includes a plurality of wires 302 braided together. The wires 302 define a plurality of pores 304 therebetween. The wires 302 also form an elongated sleeve 306 that defines an interior region 308.

The embolization device 300 includes a plurality of fibers 312. The fibers 312 are partially disposed in the pores 304. The fibers 312 increase the surface area of the embolization device 300. As such, including the fibers 312 in the embolization device 300 may improve the rate at which blood clots are formed.

The plurality of fibers 312 may exhibit an average outer dimension (e.g., diameter) that is about 10 nm to about 200 nm, about 100 nm to about 300 nm, about 200 nm to about 500 nm, about 400 nm to about 800 nm, about 700 nm to about 1.25 μm, about 1 μm to about 2 μm, about 1.5 μm to about 4 μm, about 4 μm to about 8 μm, about 6 μm to about 10 μm, about 8 μm to about 15 μm, about 10 μm to about 20 μm, about 15 μm to about 25 μm, about 20 μm to about 30 μm, about 25 μm to about 40 μm, about 30 μm to about 50 μm, about 40 μm to about 60 μm, about 50 μm to about 70 μm, about 60 μm to about 80 μm, about 70 μm to about 90 μm, about 80 μm to about 100 μm, or about 90 μm to about 125 μm. The average outer dimension of the plurality of fibers 312 may be selected based on a number of factors. In an example, the average outer dimension of the plurality of fibers 312 are selected to be smaller than the average size of the pores 304 thereby allowing at least one fiber 312 to be disposed in the pores 304. In particular, the average outer dimension of the fibers 312 may be selected to be significantly smaller than the average size of the pores 304 thereby allowing a plurality of fibers 312 to be disposed in the pores 304 which significantly increases the surface area of the embolization device 300. In an example, the average outer dimension of the plurality of fibers 312 is selected to be as small as economically and logistically possible since decreasing the average outer dimension of the fibers 312 increases the surface area of the embolization device 300 and allows the fibers 312 to be sufficiently flexible that the fibers 312 have negligible effect of the elongated and deployed configurations of the elongated sleeve 306. The plurality of fibers 312 may exhibit monofilament and/or multifilament construction.

The fibers 312 may disposed in at least some of the pores 304 and secured to the elongate sleeve 306. The fibers 312 may be disposed in and secured to the pores 304 while the wires 302 are braided. In an embodiment, a sufficient number of the fibers 312 may be disposed in the pores 304 such that the wires 302 compress the middle portion of the fibers 312. Compressing the middle portion of the fibers 312 may cause the terminal ends of the fibers 312 to flare and/or cause the fibers 312 to exhibit an interference fit with the wires 302 thereby securing the fibers 312 to the elongated sleeve 306. In an embodiment, the fibers 312 are secured to the wires 302 using an adhesive or any other suitable technique.

Disposing the fibers 312 in the pores 304 may be an improvement over using the fibers 312 in a similarly-sized conventional embolization coil. For example, conventional embolization coils may include slots between adjacent portions of the conventional embolization coils. The fibers 312 may be secured to the conventional embolization coils by disposing the fibers in the slits. However, bending the conventional embolization coils may open such slots thereby allowing the fibers 312 to fall out of the conventional embolization coils. Meanwhile, the pores 304 do not open up similar to the slots of conventional embolization coils when the embolization device 300 bends. Thus, the fibers 312 are less likely to become detached from the embolization device 300 when the embolization device 300 bends compared to conventional embolization coils.

FIG. 4 is a pictorial view of an embolization device 400 that includes at least one end cap 414, according to an embodiment. Except as otherwise disclosed herein, the embolization device 400 is the same as or substantially similar to any of the embolization devices disclosed herein. For example, the embolization device 400 may include a plurality of wires 402 forming an elongated sleeve 406.

The elongated sleeve 406 includes a first terminal end 416 and a second terminal end 418 longitudinally spaced from the first terminal end 416. The embolization device 400 may include at least one end cap 414 that is configured to at least partially cover at least one of the first terminal end 416 or the second terminal end 418 of the elongated sleeve 406. As such, the end cap 414 prevents the wires 402 at the first terminal end 416 and/or the second terminal end 418 from sticking out and jabbing (e.g., penetrating) the walls of the body of the individual. The end cap 414 may also inhibit the unbraiding of the wires 402. It is noted that the end cap 414 may decrease the surface area of the embolization device 400 but such decrease in the surface area may be relatively negligible depending on the length of the elongated sleeve 406 and the percentage of the elongated sleeve 406 covered by the end cap 414. It is noted that the elongated sleeves disclosed herein that do not include an end cap may have the terminal ends thereof folded into the interior regions to create a more atraumatic interface, such as to minimize the terminal ends thereof sticking out and pricking or sticking into the walls of the body.

The end cap 414 may be formed from any suitable biocompatible material, such as any of the biocompatible materials disclosed herein. In an embodiment, the end cap 414 may be formed from an at least partially imaging-opaque material. An at least partially imaging-opaque material may include a material that is more opaque to imaging stimuli (e.g., radio frequencies, X-rays such as X-rays from fluoroscopic imaging, etc.) than the wires 402, such as opaque to about 50% or more, about 75% or more, or about 90% or more of one or more imaging stimuli. Examples of an at least partially image-opaque material include platinum, platinum, iridium, palladium, tantalum, gold, tungsten, or combinations thereof. Forming the end cap 414 from an at least partially imaging-opaque material may make it easier to determine a position of the embolization device 400 inside a body using conventional imaging techniques. As such, the end cap 414 may be used to determine if the embolization device 400 was correctly positioned in the body, if the embolization device remained in the correct position or became dislodged, etc.

In an embodiment, the embolization device 400 may include one or more at least partially imaging-opaque markers 420 disposed on a location of the elongated sleeve 406 that is spaced from the first terminal end 416 and the second terminal end 418. The at least partially imaging-opaque markers 420 may be substantially similar to the at least partially imaging-opaque end cap 414 except that the at least partially imaging-opaque markers 420 are spaced from the first terminal end 416 and the second terminal end 418. The at least partially imaging-opaque markers 420 may be used to determine a position of a greater portion of the embolization device 400 than if the embolization device 400 only included the end caps 414. For example, the end caps 414 only provide information about the location of the terminal ends of the embolization device 400. As such, the end caps 414 may not inform a user of the embolization device 400 (e.g., surgeon) whether the embolization device 400 sufficiently occupies the desired cavity or lumen of a body. Providing the at least partially imaging-opaque markers 420 on the embolization device 400 may provide more information that may be used to determine if the embolization device 400 sufficiently occupies the desired cavity or lumen of the body.

It is noted that the elongated sleeve 406 may be detectable using conventional imaging techniques without using the at least partially imaging-opaque end caps 414 and/or the at least partially imaging-opaque markers 420. However, the at least partially image-opaque end caps 414 and/or the at least partially imaging-opaque markers 420 improve contrast such that false detection of the embolization device 400 is minimized.

In an embodiment, the embolization device 400 may include one or more at least partially image-opaque wires (e.g., platinum wires) instead of or in addition to the at least partially imaging-opaque end caps 414 and/or the at least partially imaging-opaque markers 420. For example, one or more of the wires 402 may be formed from an at least partially imaging-opaque wire (e.g., a platinum wire). The at least partially imaging-opaque wire may make it easier to determine a position of an entirety of the embolization device 400 using conventional imaging techniques.

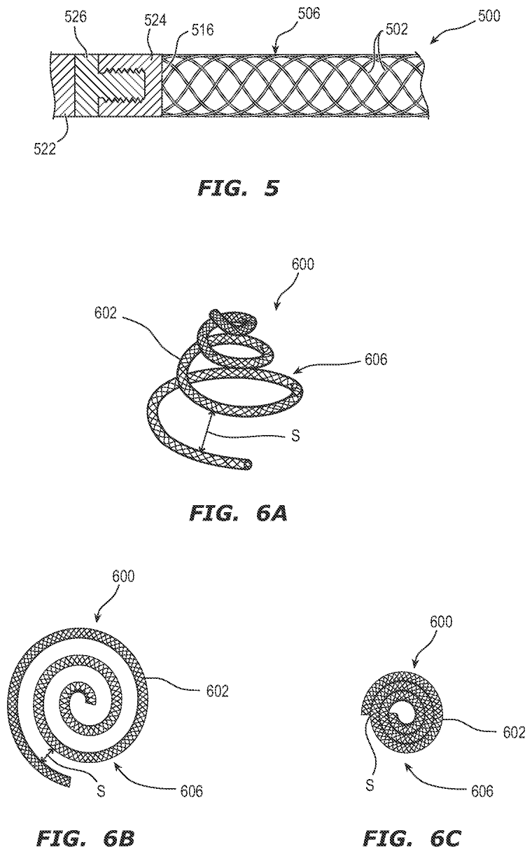

The embolization devices disclosed herein may include one or more components that facilitates positioning or the embolization devices in the body of an individual. For example, FIG. 5 is an isometric view of an embolization device 500 reversibly attached to a medical device 522 (e.g., a guidewire, delivery wire, or any other suitable medical device), according to an embodiment. Except as otherwise disclosed herein, the embolization device 500 is the same as or substantially similar to any of the embolization devices disclosed herein. For example, the embolization device 500 includes a plurality of wires 502 forming an elongated sleeve 506. The elongated sleeve 506 includes a terminal end 516.

The embolization device 500 includes a first attachment device 524 at the terminal end 516 and the medical device 522 includes a second attachment device 526. The first attachment device 524 and the second attachment device 526 are configured to be reversibly attached together. The first and second attachment devices 524, 526 allows the medical device 522 to position the embolization device 500 in a body of an individual. If the medical device 522 correctly positions the embolization device 500, the first and second attachment devices 524, 526 may be detached from each other. If the medical device 522 fails to correctly position the embolization device 500, the first and second attachment devices 524, 526 may remain attached together and the embolization device 500 may be repositioned.

The first and second attachment devices 524, 526 may include any suitable devices that may be reversibly attached together. In an example, as illustrated, the first and second attachment devices 524, 526 are threadedly attached together. In such an example, the first and second attachment devices 524, 526 may be detached from each other by rotating the medical device 522 relative to the embolization device 500. In an example, the first and second attachment devices 524, 526 may be mechanically reversibly attached together using mechanical techniques other than a threaded attachment (e.g., clamps, pins, etc.). In an example, the first and second attachment devices 524, 526 may be configured to be detached responsive to providing electrical energy to at least one of the first attachment device 524 or the second attachment device 526.

It is noted that the embolization devices disclosed herein may not include an attachment device. Instead, the embolization devices may be pushed through the catheter, for example, using a medical device (e.g., guidewire, delivery wires, etc.) or using saline solution (via syringe).

As previously discussed, the embolization devices disclosed herein may exhibit a deployed configuration. The embolization devices may exhibit any suitable shape when disposed in the deployed configuration. For example, the embolization devices may exhibit a generally spiral shape, a generally helical shape, a generally circular shape, a generally spherical shape (e.g., hollow or non-hollow spherical shape), or any other suitable shape. For brevity, the embodiments disclosed below describe the deployed configuration of the embolization devices as exhibiting a generally spiral shape. It is noted that the characteristics disclosed below regarding the generally spiral shaped deployed configuration of the embolization devices are applicable to other shapes.

FIGS. 6A and 6B are side and top plan views of an embolization device 600 is a deployed configuration, according to an embodiment. Except as otherwise disclosed herein, the embolization device 600 is the same or substantially similar to any of the embolization devices disclosed herein. For example, the embolization device 600 includes a plurality of wires 602 forming an elongated sleeve 606. Although not illustrated, the embolization device 600 may include any of the components of FIGS. 2-5.

The embolization device 600 occupies and at least partially encloses a larger volume when the embolization device 600 exhibits the deployed configuration than when the embolization device 600 exhibits the elongated configuration. The ability of the embolization device 600 to occupy and enclose a relatively large volume allows the embolization device 600 to encourage blood clotting in the relatively large volume thereby filling or blocking a cavity or lumen of the body with the blood clot. It also allows one embolization device 600 to promote blood clotting in a volume that may otherwise require a plurality of conventional embolization coils to occupy when the embolization device 600 and each of the plurality of conventional embolization coils exhibit substantially the same size.

In an embodiment, adjacent portions of the embolization device 600 may exhibit a spacing s therebetween. In an example, the spacing s may be selected to be about 500 μm or greater, about 1 mm or greater, about 1.5 mm or greater, about 2 mm or greater, about 3 mm or greater, about 4 mm or greater, about 5 mm or greater, about 6 mm or greater, about 8 mm or greater, about 10 mm or greater, about 12 mm or greater, about 15 mm or greater, or in ranges of about 500 μm to about 1.5 mm, about 1 mm to about 2 mm, about 1.5 mm to about 3 mm, about 2 mm to about 4 mm, about 3 mm to about 5 mm, about 4 mm to about 6 mm, about 5 mm to about 8 mm, about 6 mm to about 10 mm, about 8 mm to about 12 mm, or about 10 mm to about 15 mm. In an example, the spacing s may be equal or greater than the maximum lateral dimension of the elongated sleeve 606, such as about 2 or more times greater than, about 3 or more times greater than, about 4 or more times greater than, about 5 or more times greater than, about 6 or more times greater than, about 8 or more times greater than, about 10 or more times greater than, about 13 or more times greater than, or about 15 or more times greater than the maximum lateral dimension of the elongated sleeve 606. In an example, the spacing s may be sufficiently large that switching between the elongated and deployed configurations would require plastic deformation of the elongated sleeve 606. In such an example, the wires 602 may be formed from a shape memory material. Generally, increasing the spacing s increases the volume that is occupied and enclosed by the embolization device 600. However, increasing the spacing s makes it more difficult to promote blood clotting in the volume. As such, the spacing s may be selected based on balancing these two factors.

FIG. 6C is a top plan view of an embolization device 600c, according to an embodiment. The embolization device 600c is the same as the embolization device 600 illustrated in FIGS. 6A and 6B except that the spacing between adjacent portions of the embolization device 600 is substantially zero (e.g., 500 μm or less). In other words, the adjacent portions of the embolization device 600c may be touching. Selecting the spacing between adjacent portions of the embolization device 600c to be substantially zero may allow the embolization device 600c to occupy a greater percentage of a volume than the embolization device 600.

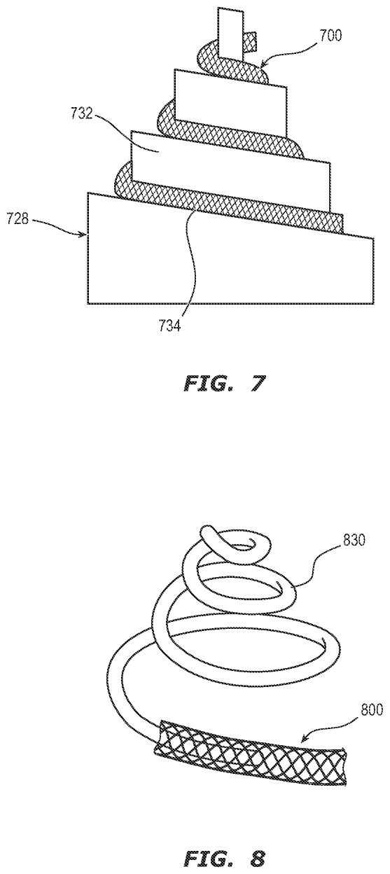

The shape of the elongated sleeve when the elongated sleeve is in the deployed configuration may be formed using any suitable technique. FIGS. 7 and 8 are pictorial views of two example methods of shaping the elongated sleeves of embolization devices such that the elongated sleeves exhibit a selected shape when in the deployed configuration, according to different embodiments. It is noted that the embolization devices illustrated in FIGS. 7 and 8 may include any of the embolization devices disclosed herein.

Referring to FIG. 7, a former 728 (e.g., a mold) may be provided. The former 728 includes one or more surfaces that the embolization device 700 may be disposed on. The embolization device 700 may be disposed on the former 728 while forming the embolization device 700 (e.g., the wires of the embolization device 700 are braided around the former 728) or after forming the embolization device 700. After disposing the embolization device 700 on the former 728, the shape of the embolization device 700 may be set such that the embolization device 700 maintains the shape that the embolization device 700 exhibits when disposed on the former 728. For example, if the embolization device 700 includes a shape memory material, setting the shape of the embolization device 700 may include heating the embolization device 700 and the former 728 to a temperature that is sufficiently high to cause the embolization device 700 to be heat set in the shape that the embolization device 700 exhibits when disposed on the former 728. The embolization device 700 may be removed from the former 728 after the embolization device 700 is able to maintain the shape that the embolization device 700 exhibits when disposed on the former 728.

As shown, the embolization device 700 exhibits a generally spiral shape. A variety of different formers 728 may be used to form the embolization device 700 into the spiral shape. In an embodiment, the former 728 includes a generally horizontal spirally-extending surface 730 and a generally vertical spirally-extending surface 732 extending from the generally horizontal spirally-extending surface 730. The embolization device 700 may be disposed in the depression formed by the intersection of the generally horizontal spirally-extending surface 730 and the generally vertical spirally-extending surface 732 thereby forming the embolization device 700 into a generally spiral shape. In another embodiment, the former 728 may exhibit a conical shape and the embolization device 700 may be wrapped around the conical former 728 such that the embolization device 700 exhibits the helical shape.

Referring to FIG. 8, a shaping wire 830 is provided. The shaping wire 830 generally exhibits the general shape of the deployed configuration of the embolization device 800. The embolization device 800 is threaded along the shaping wire 830 such that the embolization device 800 exhibits the general shape of the shaping wire 830. The shaping wire 830 may also exhibit a rigidity that prevents the shape of the shaping wire 830 from changing as the embolization device 800 is threaded along the shaping wire 830. Similar to the embolization device 700 of FIG. 7, the embolization device 800 may have the shape thereof set after threading the embolization device 800 on the shaping wire 830. In an embodiment, the embolization device 800 may be removed from the shaping wire 830 after the embolization device 800 is able to maintain the shape that the embolization device 800 exhibits when disposed on the shaping wire 830. In an embodiment, the embolization device 800 is not removed from the shaping wire 830. In such an embodiment, the shaping wire 830 forms a mandrel.

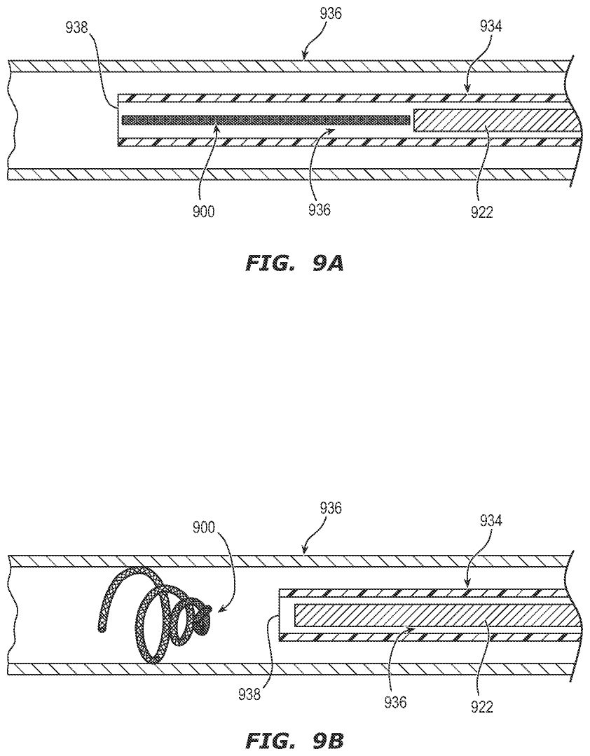

FIGS. 9A and 9B are partially cross-sectional views of a method of using any of the embolization devices disclosed herein, according to an embodiment. Referring to FIG. 9A, a medical device 932 (e.g., catheter) is disposed in a lumen 934 of a body. The lumen 934 may include a vein, an aneurysm bulge, or any other lumen or cavity of a body. An embolization device 900 exhibiting the elongated configuration thereof may be disposed in an interior passageway 936 of the medical device 932. A compressive load applied from the medical device 932 to the embolization device 900 may maintain the embolization device 900 in the elongated configuration thereof. The embolization device 900 may exhibit a generally linear shape (as shown), a generally sinusoidal shape, a generally helical shape, or any other suitable shape when in the elongated configuration. The embolization device 900 may be moved (e.g., pushed) in the interior passageway 936 towards an outlet 938 of the medical device 932. The embolization device 900 may be moved towards the outlet 938 of the medical device 932 using any of the methods disclosed herein. For example, as illustrated, the embolization device 900 may be pushed towards the outlet 938 of the medical device 932 using a delivery wire 922.

Referring to FIG. 9B, the embolization device 900 may be dispensed from the medical device 932. Dispensing the embolization device 900 from the medical device 932 removes the compressive load applied from the medical device 932 to the embolization device 900. As such, the embolization device 900 may switch from the elongated configuration to the deployed configuration thereof. The embolization device 900 may switch from the elongated configuration to the deployed configuration due to the elasticity thereof or, when the embolization device 900 includes a shape memory material, due to the heat from the body changing the embolization device 900 to its heat set shape. When in the deployed configuration thereof, the embolization device 900 may substantially occupy a portion of the lumen 934. In an example, in the illustrated embodiment, the embolization device 900 may extend between opposing surfaces of the lumen 934 such that the blood clots formed on the embolization device 900 results in complete blockage of the lumen 934. In an example, the embolization device 900 may increase a volume occupied thereby or increase the cross-section area covered thereby when the embolization device 900 switches from the elongated configuration to the deployed configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean±10%, ±5%, or +2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

What is claimed is:

1. An embolization device, comprising:
a plurality of wires braided together to form an elongated sleeve defining an interior region;
wherein the elongated sleeve is configured to switch between an elongated configuration and a deployed configuration.

2. The embolization device of claim 1, wherein the plurality of wires includes 15 wires or more.

3. The embolization device of claim 1, wherein the plurality of wires is at least partially formed from one or more shape memory materials.

4. The embolization device of claim 1, wherein the plurality of wires exhibit an average lateral dimension of about 20 μm to about 75 μm.

5. The embolization device of claim 1, wherein the elongated sleeve exhibits an outer lateral dimension of about 2 mm to about 4 mm.

6. The embolization device of claim 1, wherein the elongated sleeve exhibits an outer lateral dimension of about 1 mm or less.

7. The embolization device of claim 1, wherein the deployed configuration of the elongated sleeve exhibits a generally spiral shape.

8. The embolization device of claim 1, further comprising at least one end cap attached to a terminal end of the elongated sleeve, the at least one end cap band formed from an at least partially image-opaque material.

9. The embolization device of claim 1, further comprising a first attachment device attached to the elongated sleeve, the first attachment device configured to be reversibly attached to a medical device.

10. The embolization device of claim 1, wherein the plurality of wires define a plurality of pores therebetween and, further comprising a plurality of fibers disposed in at least some of the plurality of pores.

11. The embolization device of claim 1, further comprising a mandrel disposed in the interior region.

12. A method of making an embolization device, the method comprising:
braiding a plurality of wires together to form an elongated sleeve defining an interior region;
wherein the elongated sleeve is configured to switch between an elongated configuration and a deployed configuration.

13. The method of claim 12, wherein braiding the plurality of wires includes braiding the plurality of wires without using a mandrel.

14. The method of claim 12, wherein braiding the plurality of wires includes braiding the plurality of wires with a mandrel disposed in the interior region.

15. The method of claim 12, further comprising, after braiding the plurality of wires, stretching the elongated sleeve to draw down a maximum lateral dimension of the elongated sleeve.

16. The method of claim 12, further comprising, after braiding the plurality of wires, wrapping the elongated sleeve around a former to create the deployed configuration of the elongated sleeve.

17. A method of using an embolization device, the method comprising:
dispensing the embolization device from an outlet of a medical device and into a lumen of body, the embolization device including a plurality of wires braided together to form an elongated sleeve defining an interior region, wherein the elongated sleeve exhibiting an elongated configuration when disposed in the catheter; and
switching the elongated sleeve of the embolization device from the elongated configuration to a deployed configuration responsive to dispensing the embolization device from the medical device.

18. The method of claim 17, wherein the embolization device includes a first attachment devices reversibly attached to a second attachment device; and
further comprising, after dispensing the embolization device from the outlet, detaching the first attachment device from the second attachment device.

19. The method of claim 17, wherein dispensing the embolization device from the outlet includes pushing the embolization device out of the medical device using a delivery wire, wherein the embolization device is not reversibly attached to the guidewire.

20. The method of claim 17, wherein switching the elongated sleeve of the embolization device from the elongated configuration to a deployed configuration includes at least one of increasing a volume occupied by the elongated sleeve or increasing the cross-sectional area covered by the elongated sleeve.
